SEQUENCE LISTING (12 sequences)

SEQ ID NO: 1 - DNA, 22 bp, Artificial Sequence
  hsa-miR-214-3p MIMAT0000271
acagcaggcacagacaggcagu

SEQ ID NO: 2 - DNA, 22 bp, Artificial Sequence
  hsa-miR-34c-3p MIMAT0004677
aaucacuaaccacacggccagg

SEQ ID NO: 3 - DNA, 22 bp, Artificial Sequence
  NCKAP1-3′ UTR
ttagtgattggtgtgccggtcc

SEQ ID NO: 4 - DNA, 23 bp, Artificial Sequence
  NCKAP1-3′ UTR
agtgattattttagatagtgata

SEQ ID NO: 5 - DNA, 22 bp, Artificial Sequence
  hsa-miR-34c-3p
ggaccggcacaccaaucacuaa

SEQ ID NO: 6 - DNA, 23 bp, Artificial Sequence
  NCKAP1-3′UTR
gttgagcttggattttagtgatg

SEQ ID NO: 7 - DNA, 22 bp, Artificial Sequence
  hsa-miR-34c-3p
ggaccggcacaccaaucacuaa

SEQ ID NO: 8 - DNA, 22 bp, Artificial Sequence
  NCKAP1-3′UTR
gcactaccattgtacctgctgt

SEQ ID NO: 9 - DNA, 21 bp, Artificial Sequence
  hsa-miR-214-3p
gacggacagacacggacgaca

SEQ ID NO: 10 - RNA, 22 nt, Artificial Sequence
  miR-214-3p mimic
ugccugucuacacuugcugugc

SEQ ID NO: 11 - RNA, 21 nt, Artificial Sequence
  miR-34c-3p mimic
aaucacuaaccacacggcagg

SEQ ID NO: 12 - RNA, 22 nt, Artificial Sequence
  miR-214-5p inhibitor
ugccugucuacacuugcugugc

(19) United States
(12) Patent Application Publication
(10) Pub. No.: US 2025/0270642 A1
(43) Pub. Date: Aug. 28, 2025

(54) BIOMARKER FOR PREDICTING PROGNOSIS OF NEURODEGENERATIVE DISEASES INCLUDING miRNA AND USE THEREOF

(71) Applicant: IUCF-HYU (INDUSTRY-UNIVERSITY COOPERATION FOUNDATION HANYANG UNIVERSITY), Seoul (KR)

(72) Inventors: Seung Hyun KIM, Seoul (KR); Min Young NOH, Seoul (KR)

(73) Assignee: IUCF-HYU (INDUSTRY-UNIVERSITY COOPERATION FOUNDATION HANYANG UNIVERSITY), Seoul (KR)

(21) Appl. No.: 18/290,378

(22) PCT Filed: May 13, 2022
(86) PCT No.: PCT/KR2022/006918

§ 371 Date: May 8, 2024

(30) Foreign Application Priority Data
May 14, 2021 (KR) 10-2021-0062992

(51) Int. Cl. C12Q 1/6883

(52) U.S. Cl. CPC C12Q 1/6883; C12Q 2600/118; C12Q 2600/158; C12Q 2600/178

(57) ABSTRACT

The present invention relates to a biomarker to predict the prognosis of neurodegenerative diseases, and a use thereof, and, more particularly, to: a marker composition for prognosis prediction of neurodegenerative diseases, comprising miRNA-214 and/or miRNA-34c; a composition and a kit for prognosis prediction of neurodegenerative diseases, comprising an agent for measuring the expression level of the miRNAs; and a method for predicting the prognosis of neurodegenerative diseases by using the marker. It has been identified that the miRNA-214 and/or miRNA-34c according to the present invention can regulate phagocytic capacity modulator of microglia, NKCAP1 so that the prognosis of neurodegenerative diseases can be predicted, and thus it is expected that the present invention can be effectively used as a biomarker for predicting the prognosis of neurodegenerative diseases.

### TECHNICAL FIELD

The present disclosure relates to a marker composition to predict prognosis of neurodegenerative diseases, the marker composition containing miRNA-214 and miRNA-34c capable of regulating the expression of the NCKAP1 gene which is a phagocytic factor of microglia. In addition, the present disclosure relates to a kit to predict prognosis of neurodegenerative diseases, and to a method of providing information.

### BACKGROUND ART

Neurodegenerative diseases are diseases that cause abnormalities in motor control ability, cognitive function, perceptual function, sensory function, and autonomic nerve function due to loss of nerve structure and function. As the population is rapidly aging around the world, the number of patients with neurodegenerative diseases such as Amyotrophic lateral sclerosis (ALS), Alzheimer's disease (AD), Parkinson's disease (PD), and Frontotemporal dementia (FTD) is increasing rapidly. These neurodegenerative diseases often leave sequela since nerve cells have the characteristics of having difficulties in repairment and regeneration once damaged or killed, thereby patients do not return to normal and the diseases continue to worsen. Therefore, research has been actively conducted at home and abroad for several decades to solve these problems, but the exact cause of the diseases has not been properly identified. Because of these characteristics, most neurodegenerative diseases cannot be cured with current treatment techniques, and the focus is mainly on targeting neuroinflammation and transmission processes to regulate the progression rate and alleviate symptoms of the diseases. Therefore, there is an urgent need to develop new treatment targets and treatments for neurodegenerative diseases, and it is also an important task to discover markers that can diagnose the diseases earlier and predict prognosis.

In the past, the proliferation of neuroglial cells and infiltration of cells related to immune inflammation and immune mediators observed in pathological findings over neurodegenerative diseases have been accepted only as a non-specific secondary phenomenon resulting from the destruction of nerve cells. However, with the advance of molecular biology, it became possible to identify epidermal markers of cells that play an important role in the immune-inflammatory mechanism. Evidence suggests as the identity and functions of the immune-inflammatory mediators are identified, the immune-inflammatory mechanism is not only a non-specific secondary response to damage to the nervous system, but may also be involved early in the progression of neurodegenerative diseases, may regulate the pace of pathology, and may be involved in the survival and death of nerve cells.

Meanwhile, microglia are a main cell that affects neuroinflammation in neurodegenerative diseases, and microglia are involved in neuronal integrity and death of nerve cells in brains by remodeling synapses, secreting various nerve growth factors, and removing various debris that accumulate in the brain, thereby microglia play an important role in maintaining biological homeostasis. According to recent papers, the functions of the microglia are well-regulated under a normal condition, but under a neurodegenerative disease condition, microglia are activated by various unfolded or aggregated proteins (for example, amyloid-β causing Alzheimer's disease, α-synuclein causing Parkinson's disease, SOD1 causing ALS, and Tau, TDP-43, and Annexin All causing Frontotemporal dementia), as well as the numerical changes and functional abnormalities of the microglia act as a stimulant of the inflammatory response, causing neuroinflammation, thereby affecting the occurrence and progression of neurodegenerative diseases. Thus, microglial loss or dysfunction is being emphasized in treating neurodegenerative diseases. Therefore, in addition to existing treatment methods, a focus is on treating neurodegenerative diseases through modulating the functions of the microglia.

MicroRNAs (miRNAs) are a small-RNA molecule consisting of about 22 nucleotides and regulate gene expression through the destruction of target mRNA or inhibition of the target mRNA at the translation stage. The miRNAs are involved in various physiological phenomena and diseases, and in the central nervous system. Loss of Dicer which is a key regulator of miRNA production, induces neurodegeneration, showing that balanced expression of miRNAs plays an important role in the nervous system. The miRNAs are detectable at stable levels in blood and other body fluids and are used for disease diagnosis or prognosis. To date, many studies have reported that the miRNAs are differentially expressed in ALS patients compared to the control group in various biological fluids, including CSF, plasma containing blood-derived components, and plasma, and the studies expanded our understanding of the pathogenesis of ALS. However, utilizing the miRNAs for disease prognosis prediction and direct treatment in ALS has not yet been attempted.

Therefore, the present disclosure seeks to develop a biomarker that can predict the prognosis of neurodegenerative diseases using miRNAs in the blood that regulate the expression of the NCKAP1 gene that is a modulator of microglial phagocytic capacity under a neurodegenerative disease condition.

### DISCLOSURE

### Technical Problem

Against the background, the present inventors have made efforts to discover biomarkers that can predict prognosis of neurodegenerative diseases and confirmed that the miRNAs of the present disclosure can predict the prognosis of neurodegenerative diseases by regulating the expression of the NCKAP1 gene that is a modulator of microglial phagocytic capacity, and the inventors completed the present disclosure.

Accordingly, one objective of the present disclosure is to provide a marker composition for predicting prognosis of neurodegenerative diseases, and the marker composition contains at least one selected from the group consisting of miRNA-214 and miRNA-34c.

Another object of the present disclosure is to provide a composition for predicting the prognosis of neurodegenerative diseases, and the composition contains a miRNA expression measuring agent for measuring an expression level of at least one miRNA selected from the group consisting of miRNA-214 and miRNA-34c.

Another objective of the present disclosure is to provide a kit for predicting the prognosis of neurodegenerative diseases, and the kit contains the composition.

In addition, another objective of the present disclosure is to provide a method of providing information to predict prognosis of neurodegenerative diseases, and the method includes measuring the expression level of at least one selected from the group consisting of miRNA-214 and miRNA-34c in a biological sample derived from a subject.

In addition, the other objective of the present disclosure is to provide a method of predicting the prognosis of neurodegenerative diseases, and the method includes measuring or detecting the expression level of at least one selected from the group consisting of miRNA-214 and miRNA-34c in a biological sample derived from a subject.

However, the technical problems to be achieved by the present disclosure are not limited to the problems mentioned above, and other problems not mentioned will be clearly understood by those skilled in the art from the description below.

### Technical Solution

To achieve the objectives of the present disclosure as described above, the present disclosure provides a marker composition for predicting prognosis of neurodegenerative diseases, and the marker composition contains at least one selected from the group consisting of miRNA-214 and miRNA-34c.

In one embodiment of the present disclosure, the miRNA-214 may be represented by the base sequence of SEQ ID NO: 1.

In another embodiment of the present disclosure, the miRNA-34c may be represented by the base sequence of SEQ ID NO: 2.

In another embodiment of the present disclosure, neurodegenerative diseases may be selected from the group consisting of Parkinson's disease, dementia, Alzheimer's disease, Frontotemporal dementia, Huntington's disease, and ALS.

In another embodiment of the present disclosure, the miRNA-214 and miRNA-34c may inhibit expression of NCKAP1 (Nck-associated protein 1).

In addition, the present disclosure provides a composition for predicting the prognosis of neurodegenerative diseases, and the composition contains an agent for measuring an expression level of at least one miRNA selected from the group consisting of miRNA-214 and miRNA-34c.

In one embodiment of the present disclosure, the agent for measuring the expression level of the miRNA may be a sense and antisense primer or a probe that binds to the miRNA complementarily.

In another embodiment of the present disclosure, the miRNA-214 may be represented by the base sequence of SEQ ID NO: 1.

In another embodiment of the present disclosure, the miRNA-34c may be represented by the base sequence of SEQ ID NO: 2.

In another embodiment of the present disclosure, the neurodegenerative diseases may be selected from the group consisting of Parkinson's disease, dementia, Alzheimer's disease, Frontotemporal dementia, Huntington's disease, and ALS.

Additionally, the present disclosure provides a kit for predicting the prognosis of neurodegenerative diseases, and the kit contains the composition.

In addition, the present disclosure provides a method of to predict the prognosis of providing information neurodegenerative diseases, and the method includes measuring the expression level of at least one selected from the group consisting of miRNA-214 and miRNA-34c in a biological sample derived from a subject.

In one embodiment of the present disclosure, the method of providing information further includes determining that the progression of neurodegenerative diseases is rapid when at least one selected from the group consisting of miRNA-214 and miRNA-34c exhibits a high expression level.

In another embodiment of the present disclosure, the miRNA-214 may be represented by the base sequence of SEQ ID NO: 1.

In another embodiment of the present disclosure, the miRNA-34c may be represented by the base sequence of SEQ ID NO: 2.

In another embodiment of the present disclosure, the expression levels of the miRNA-214 and miRNA-34c may be measured through at least one method selected from the group consisting of next generation sequencing (NGS), a polymerase chain reaction (PCR), and a reverse transcription polymerase chain reaction (RT-PCR), a Real-time polymerase chain reaction (Real-time PCR), an RNase protection assay (RPA), microarray, and northern blotting.

In addition, the present disclosure provides a method of predicting the prognosis of neurodegenerative diseases, and the method includes measuring or detecting the expression level of at least one selected from the group consisting of miRNA-214 and miRNA-34c in a biological sample derived from a subject.

In one embodiment of the present disclosure, the method may further include obtaining a biological sample from a subject or human patient.

### Advantageous Effects

In the present disclosure, it was confirmed that prognosis of neurodegenerative diseases could be predicted by confirming whether miRNA-214 and miRNA-34c (which are miRNAs capable of regulating NCKAP1 serving as a modulator of microglial phagocytic capacity) were expressed in microglia and plasma sample.

Since the miRNAs can predict prognosis in patients with neurodegenerative diseases, the miRNAs are expected to be useful in predicting prognosis and establishing customized treatment strategies for patients.

However, the effects of the present disclosure are not limited to the effects and should be understood to include all effects that can be inferred from the configuration of the disclosure described in the description or claims of the present disclosure.

### DESCRIPTION OF DRAWINGS

FIG. 1a shows the binding sites of each of miRNA-214-3p and miRNA-34c-3p to the NCKAP1 gene;

FIG. 1b shows changes in the protein level of NCKAP1 when NCKAP1 was treated with each of the mimic and inhibitor of miRNA-214-3p and treated with each of the mimic and inhibitor of miRNA-34c-3p;

A and B in FIG. 1c show the results of analyzing the correlation between the expression level of each of the miRNAs of the present disclosure and the expression level of NCKAP1 miRNA, as measured in induced microglia-like cells (iMGs) and plasma derived from patients with ALS, respectively, and specifically A in FIG. 1c shows the results of confirming the correlation between miRNA-214-3p and NCKAP1, and B in FIG. 1c shows the results of confirming the correlation between miRNA-34c-3p and NCKAP1;

FIGS. 2A and 2B show the results of measuring the expression level of the miRNAs in induced microglia-like cells (iMGs) depending on the progression rate of patients with ALS, and specifically FIG. 2A shows the results of confirming the expression level of miRNA-214-3p, and FIG. 2B shows the results of confirming the expression level of miRNA-34c-3p;

FIGS. 3A and 3B show the results of measuring miRNA expression levels in patient's depending on the progression rate of the patients with ALS, and specifically FIG. 3A shows the results of confirming the expression level of miRNA-214-3p, and FIG. 3B shows the results of confirming the expression level of miRNA-34c-3p;

FIG. 4 shows the results of confirming the correlation between the expression of miRNA-214-3p and the expression of miRNA-34c-3p (which are the present disclosure's miRNAs), as measured in induced microglia-like cells (iMGs) and plasma derived from the patients with ALS, respectively;

FIGS. 5A and 5B show the results of confirming the correlation between the progression rate of ALS and the expression of each of the present disclosure's miRNAs by comparing the expression level of each of the miRNAs in the plasma derived from the patients with ALS and the delta-FS values, and especially A in FIG. 5 shows the results of confirming the correlation between the delta-FS value and expression of miRNA-214-3p, and B in FIG. 5 shows the results of confirming the correlation between the delta-FS value and expression of miRNA-34c-3p;

FIGS. 6A and 6B show the results of re-validating (in validation set) the correlation between the expression level of miRNA-214-3p in the plasma derived from the patients with ALS and the progression rate of ALS, and especially FIG. 6A shows the results of reconfirming the expression level of miRNA-214-3p in each of the progression rate-specific groups of the patients with ALS, and FIG. 6B shows the results of comparing the expression level of miRNA-214-3p with delta-FS to reconfirm the correlation between the expression level of miRNA-214-3p and the progression rate in patients with ALS.

### MODE FOR DISCLOSURE

The present inventors have found that prognosis of degenerative brain diseases could be predicted by expressing miRNA-214 and miRNA-34c in microglia and plasma, respectively, the miRNA-214 and miRNA-34c being miRNAs that can regulate the expression of the NCKAP1 gene that is a modulator of microglial phagocytic capacity. As confirmed, the present disclosure has been completed.

Accordingly, the present disclosure provides a marker composition for predicting the prognosis of neurodegenerative diseases, and the marker composition contains at least one selected from the group consisting of miRNA-214 and miRNA-34c.

In addition, the present disclosure provides a composition for predicting the prognosis of neurodegenerative diseases, and the composition contains a miRNA expression measuring agent for measuring an expression level of at least one miRNA selected from the group consisting of miRNA-214 and miRNA-34c. The present disclosure also provides a kit containing the composition for predicting the prognosis of neurodegenerative diseases.

The miRNA-214, as discovered herein, is a biomarker to predict prognosis of neurodegenerative diseases and may be represented by the nucleic sequence of SEQ ID NO: 1. At this time, the miRNA-214 may have nucleic sequences having sequence homology of at least 70%, preferably at least 80%, more preferably at least 90%, and most preferably at least 95% to the nucleic sequence represented by SEQ ID NO: 1.

The miRNA-34c, as discovered herein, is a biomarker to predict prognosis of neurodegenerative diseases and may be represented by the nucleic sequence of SEQ ID NO: 2. At this time, the miRNA-34c may have nucleic sequences having sequence homology of at least 70%, preferably at least 80%, more preferably at least 90%, and most preferably at least 95% to the nucleic sequence represented by SEQ ID NO: 2.

The term “neurodegenerative diseases”, as used herein, refers to Parkinson's disease, dementia, Alzheimer's disease, Frontotemporal dementia, Huntington's disease, stroke, cerebral infarction, Pick's disease, head trauma, spinal cord injury, cerebral arteriosclerosis, ALS, and multiple sclerosis, geriatric depression, or Creutzfeldt-Jakob disease. The term preferably refers to Parkinson's disease, dementia, Alzheimer's disease, Frontotemporal dementia, Huntington's disease, or ALS, and more preferably ALS or Alzheimer's disease but is not limited to the mentioned diseases.

The term “NCKAP1 (Nck-associated protein 1)”, as used herein, is a modulator of microglial phagocytic capacity that can cause neurodegenerative diseases when dysfunction of NCKAP1 occurs.

The term “prognosis”, as used herein, refers to the prediction of the progression, progress, and recovery of the diseases, and may mean the identification of metastasis, progression rate, disease-free survival rate, and survival rate, and preferably the progression rate of the diseases.

In the present disclosure, the agent for measuring the expression level of the miRNA-214 or miRNA-34c may be a sense and antisense primer or a probe that binds complementary to each of the miRNAs but is not limited to the mentioned above.

The term “primer”, as used herein, refers to a short gene sequence that serves as the starting point for DNA synthesis and an oligonucleotide synthesized for use in diagnosis and DNA sequencing. The primers can typically be synthesized and used in a length of 15 to 30 base pairs but may vary depending on the purpose of use, and can be modified by known methods such as methylation and capping.

The term “probe”, as used herein, refers to a nucleic acid that can specifically bind to mRNA with a length of a few bases to hundreds of bases, the mRNA being produced through chemical enzyme isolation and the process of purification or synthesis. The probe can be used to confirm the presence or absence of mRNA by labeling mRNA with radioactive isotopes, enzymes, or fluorescent substances and can be designed, modified, and used by known methods.

The diagnostic kit has at least one of compositions containing different components, solutions, or devices suitable for analysis methods.

For example, to perform PCR, the kit may contain genomic DNA derived from a sample to be analyzed, a primer set specific for the marker gene of the present disclosure, an appropriate amount of DNA polymerase, dNTP mixture, PCR buffer solution, and water. The PCR buffer solution may contain KCl, Tris-HCl, and MgCl₂. In addition, the kit may further contain components necessary for performing electrophoresis to confirm the amplification of the PCR product.

Additionally, the kit may be a kit containing essential elements required to perform RT-PCR. In addition to each primer pair specific for the marker gene, the RT-PCR kit contains test tubes or other suitable containers, reaction buffer, deoxynucleotides (dNTPs), enzymes such as Taq-polymerase and reverse transcriptase, DNase, RNase inhibitors, and DEPC-water, and sterilized water. The RT-PCR kits may also contain primer pairs specific for genes used as a quantitative control.

Through specific examples, the present inventors have confirmed that the miRNA of the present disclosure could be used as an indicator to predict the progression rate of the diseases in patients with neurodegenerative diseases.

More specifically, in one example of the present disclosure, it was confirmed the progression rate of the diseases in patients with neurodegenerative diseases could be predicted through the expression level of miRNA-214 or miRNA-34c which are the miRNAs of the present disclosure (see Example 3).

In another example of the present disclosure, re-verification was performed to confirm the prognostic effect of the present disclosure's miRNAs in predicting neurodegenerative diseases. At this time, it was possible to verify again that the progression rate of the diseases in patients with neurodegenerative diseases could be predicted by confirming the expression levels of the miRNAs which are miRNA-214 and miRNA-34c (see Example 4).

Therefore, the results demonstrate the prognostic effect and use of the miRNAs for patients with neurodegenerative diseases.

Accordingly, in another aspect of the present disclosure, the present disclosure provides a method of providing information to predict prognosis of neurodegenerative diseases, and the method includes measuring the expression level of at least one selected from the group consisting of miRNA-214 and miRNA-34c in a biological sample derived from a subject.

The biological sample derived from the subject may include tissue, cells, whole blood, blood, saliva, sputum, cerebrospinal fluid, or urine and preferably may be cells or plasma, but the sample is not limited to the mentioned above.

In the present disclosure, when the expression level of miRNA-214, miRNA-34c, or both in the sample derived from the subject according to the methods is higher than the expression level of miRNA-214, miRNA-34c, or both in the sample derived from the patients with neurodegenerative diseases in the comparison group, it can be determined that the progression of neurodegenerative diseases in the subject is faster than that in the comparison group of the patients.

The expression level of the miRNAs can be measured by at least one conventional method known in the art selected from the group consisting of next generation sequencing (NGS), polymerase chain reaction (PCR), reverse transcription polymerase chain reaction (RT-PCR), real-time polymerase chain reaction, RNase protection assay (RPA), microarray, and northern blotting, but the measurement method is limited to the mentioned methods.

In addition, another objective of the present disclosure is to provide a method of predicting the prognosis of neurodegenerative diseases, and the method includes measuring or detecting the expression level of at least one selected from the group consisting of miRNA-214 and miRNA-34c in a biological sample derived from a subject.

In the present disclosure, the method may further include obtaining a biological sample from a subject or human patient.

Hereinafter, preferred examples are proposed to help understand the present disclosure. However, the following examples are provided only to make the present disclosure easier to understand, and the content of the present disclosure is not limited by the following examples.

### EXAMPLES

### Example 1. Method & Materials

### 1-1. Recruitment of Analysis Subjects

To discover miRNAs capable of predicting the prognosis of neurodegenerative diseases, a method of inducing microglia was used using blood from patients with ALS which was one of neurodegenerative diseases. Medical data from HY-ALS was about 5 healthy volunteers and 29 patients with ALS (14 patients with slow progression and 15 patients with rapid progression) who were subjects of this trial. The data was obtained during the period between September 2015 and July 2017, and the average value of progression rate (ΔFS=0.75) shown in the medical data was set as a standard. When the patients had FS values above the standard, the patients were classified into the rapid progression group (Rapid-ALS), and when the patients had FS values below the standard, the patients were classified into the slow progression group (Slow-ALS). By reviewing the patient's medical records over a follow-up period of one year or more, age, gender, ALS family history, symptom onset, disease duration, and ALS functional rating scale-revised (ALSERS-R) scores were obtained, and specific information is shown in Table 1 below.

TABLE 1

| | Rapid- | | | | | |
|---|---|---|---|---|---|---|
| Characteristic | ALS | | Slow-ALS | | Controls | |
| Number of subjects | 15 | | 14 | | 5 | |
| Male:Female, n | 9:6 | | 8:6 | | 3:2 | |
| Onset age, yr | 55.6 | (6.0) | 49.9 | (6.6) | — | |
| Age at diagnosis, yr | 56.2 | (6.1) | 52.4 | (7.0) | | |
| ALSFRS-R at diagnosis | 36.5 | (3.8) | 42.4 | (3.7) | — | |
| Age at sampling time for iMG, yr | 56.7 | (5.9) | 54.6 | (8.1) | 32.0 | (4.8) |
| ALSFRS-R at sampling time | 27.7 | (7.6) | 37.9 | (8.3) | — | |
| for iMG model | | | | | | |
| Disease duration (from onset to | 13.7 | (6.2) | 53.0 | (54.9) | — | |
| sampling time)^{a}, mo | | | | | | |
| Delta-FS (from onset to sampling | 1.6 | (0.6) | 0.2 | (0.1) | — | |
| time)^{b}, point/mo | | | | | | |
| Family history of amyotrophic | 0 | (0%) | 0 | (0%) | 0 | (0%) |

lateral sclerosis, n (%)

Data were expressed as mean (standard deviation) for continuous variables, and n (%) was expressed for categorical variables.

^{a} Disease duration: The period from disease onset to blood collection.

^{b}The level of progression was defined as delta FS (48 (at diagnosis)−ALSFRS-R score)/(time from onset to diagnosis (months)), and the patients with ALS were categorized according to the following criteria: slowly progressing group (Slow, delta FS≤0.75), rapidly progressing group (Rapid, delta FS>1.0).

1-2. Manufacturing of Induced Microglia-Like Cells (iMGs)

The manufacturing of induced microglia-like cells was performed using the previously published method of Ohganani (Ohgidani M, et al. Sci Rep 2014, 4:4957.). Specifically, blood was collected from the patients in Example 1-1, and then peripheral blood mononuclear cells (PBMC) were isolated through density gradient centrifugation using Ficoll (GE Healthcare, Uppsala, Sweden). The peripheral blood mononuclear cells isolated in this way were incubated for one day at a density of 500,000 cells/ml in RPMI-1640 medium under standard culture conditions (37° C., 5% CO₂) containing 10% of Fetal Bovine Serum (FBS) and 1% antibiotic/antimycotic solution (Invitrogen, Carlsbad, CA) for modeling of microglia. Afterward, adherent cells (monocytes) were incubated for 21 days in RPMI-1640 Glutamax (Gibco, Waltham, MA) supplemented with 1% of antibiotic/antimycotic solution, 10 ng/ml recombinant human granulocyte-macrophage colony-stimulating factor (GM-CSF), and 100 ng/ml recombinant human interleukin-34 (IL-34).

1-3. Manufacturing of miRNA Mimics and Inhibitors of the Present Disclosure

A mimic and inhibitor of each of miRNA-214 (miRNA-214-3) and miRNA-34c-3p were synthesized by Bioneer Corporation (Daejeon, Korea), the miRNAs being expected to be used to predict prognosis of neurodegenerative diseases because the miRNAs were capable of modulating the microglial phagocytic capacity. As a negative control, miRNA-214-5p was also manufactured and used by Bioneer Corporation.

TABLE 2

| miRNA | Primer sequence |
|---|---|
| miR-214-3p | 5′-UGCCUGUCUACACUUGCUGUGC-3′ |
| mimic | (SEQ ID NO: 10) |
| miR-34c-3p | 5′-AAUCACUAACCACACGGCAGG-3′ |
| mimic | (SEQ ID NO: 11) |
| miRNA mimic | Bioneer Corporation, Korea, |
| negative | #SMC-2003 |
| control #1 | |
| miR-214-5p | 5′-UGCCUGUCUACACUUGCUGUGC-3′ |
| inhibitor | (SEQ ID NO: 12) |
| miR-214-3p | 5′-ACAGCAGGCACAGACAGGCAGU-3′ |
| inhibitor | (SEQ ID NO: 1) |
| miR-34c-3p | 5′-AAUCACUAACCACACGGCCAGG-3′ |
| inhibitor | (SEQ ID NO: 2) |
| miRNA | Bioneer Corporation, Korea, |
| inhibitor | #SMC-2103 |
| negative | |
| control #1 | |

HeLa cells subjected to transformation with the oligonucleotides were incubated in Dulbecco's modified Eagle's medium additionally with 10% FBS (Gibco), sodium bicarbonate, sodium pyruvate (sigma), and antibiotics. In terms of the handling of the synthesized miRNA mimics, inhibitors, and negative control miRNAs, 50 nM of the miRNA mimics, inhibitors, or controls were treated to the Hela cells plated at a density of 3×10⁵ cells/well by using Lipofectamine® RNAiMAX (Invitrogen) in accordance with manufacturer's instructions, and as a result transformation was carried out.

### 1-4. Immunoblotting

In immunoblotting for protein detection, equal amounts of each of the proteins (40 μg) were isolated by 10% of SDS-PAGE and then transferred to a PVDF membrane (GE Healthcare). The PVDF membrane was blocked with 5% skim milk and then the proteins were incubated with primary antibodies. The membrane was washed with Tris-buffered saline containing 0.05% Tween-20, and the proteins were treated with HRP (horseradish peroxidase)-conjugated secondary mouse antibodies or rabbit antibodies (Amersham Pharmacia Biotech). Afterward, enhanced chemiluminescence (ECL) detection was carried out with the proteins.

1-5. Analysis of Plasma miRNA

To analyze plasma miRNAs, 625 μl of plasma sample was extracted, and the small RNA-enriched fractions were extracted using the mirVana miRNA isolation kit in accordance with the manufacturer's instructions (Ambion, Austin, TX). Endogenous controls, hsa-miR-214-3p (4427975, ID002306), and hsa-miR-34c-3p (4427975, ID 241009_mat) were prepared using Life Technologies products.

### 1-6. Quantitative Real-Time Polymerase Chain Reaction

Total RNAs were extracted using Trizol reagents (Invitrogen) and then confirmed using a NanoDrop 2000 spectrophotometer (Thermo Scientific, ND-2000). cDNAs were synthesized using a cDNA kit (Clontech, CA, USA). The cDNAs were amplified using a Power SYBR Green PCR Master Mix with primers in an Applied Biosystems Step One Plus™ system (Life Technologies) at a temperature of 95° C. for 10 minutes. Afterward, the process of amplification with the cDNAs at a temperature of 95° C. for 15 seconds and then at a temperature of 60° C. for 1 minute was set as one cycle and the cycle was repeated 40 times. A melting curve analysis was performed to observe the specificity of the amplification, and a relative quantity (RQ) level was calculated based on the 2-ΔΔCt method using GAPDH as an internal standard control group. The results of the trials were achieved based on three independent trials, and the used primers were NCKAP1 (Qiagen, pp 5666 H1A) and GAPDH (Qiagen, PPH00150F).

### 1-7. Statistical Analysis

Data were expressed as mean±standard deviation, the t-test and one-way analysis of variance were performed to identify statistical significance between groups, and Tukey's post hoc analysis was performed using Prism 9 (GraphPad Software, San Diego, CA). The significance of data was expressed as *p<0.05, ** p<0.01, and *** p<0.001, and the p-value was obtained using Spearman's rank correlation coefficient in the post hoc analysis.

### Example 2. Discovery of miRNAs to Predict Prognosis of Neurodegenerative Diseases

2-1. Confirmation of the Capacity of the Present Disclosure's miRNAs in Modulating NCKAP1

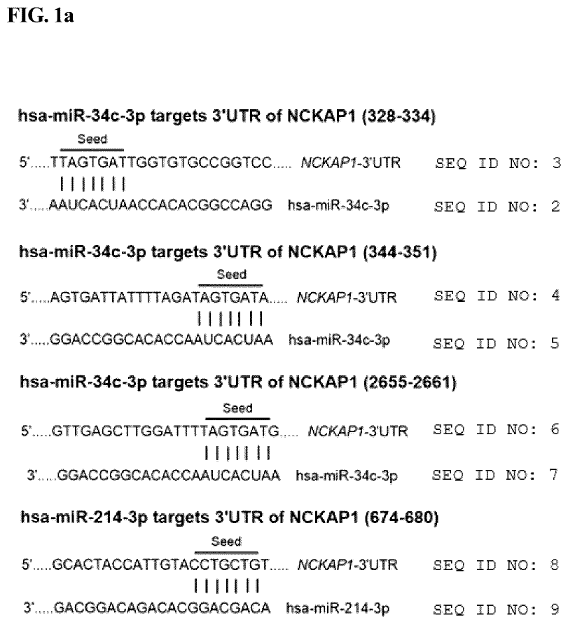

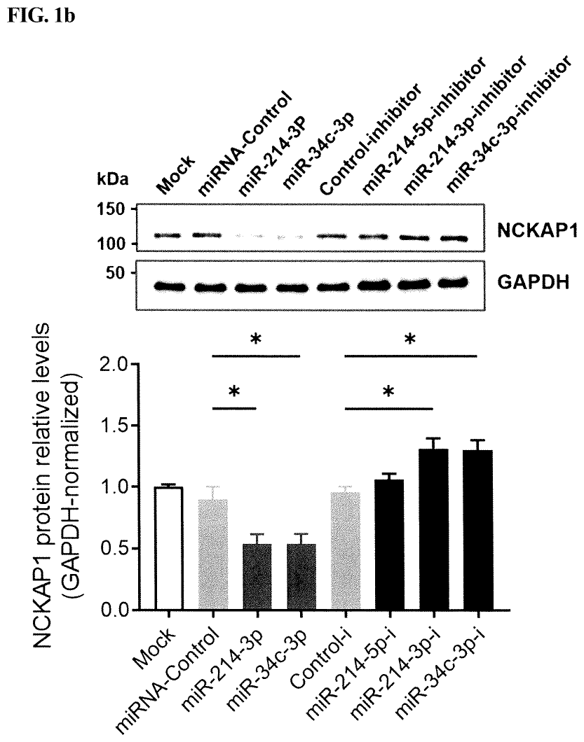

As shown in FIG. 1a, miRNA-214-3p and miRNA-34c-3p which were the present disclosure's miRNAs and expected to be able to predict the prognosis of neurodegenerative diseases by modulating the microglial phagocytic capacity were confirmed to have a seed site to modulate the phagocytic capacity of microglia by binding to NCKAP1 which served to modulate the microglial phagocytic capacity. After induced microglia-like cells were transformed with mimics (miR-214-3p and miR-34c-3p) and miRNA inhibitors (miRNA-214-5p inhibitor, miRNA-214-3p inhibitor, and miRNA-34c-3p inhibitor), the protein expression level of NCKAP1 was observed. It was confirmed that the miRNA mimics suppressed the expression level of NCKAP1, but the miRNA inhibitors could enhance the expression of NCKAP1.

In addition, to specifically confirm the results, the correlation was analyzed between the mRNA expression level of NCKAP1 serving as the modulator of microglial phagocytic capacity and the expression of each of the miRNAs, as measured in induced microglia-like cells (iMGs) and plasma derived from patients with ALS, respectively. As a result, as shown in FIG. 1c, the miRNAs showed a negative correlation not only in the induced microglia-like cells (iMGs) but also in the plasma. Through this, the present inventors were able to confirm that the effect of the miRNAs in predicting the progression rate of ALS was achieved through the modulation of NCKAP1 serving as the modulator of microglial phagocytic capacity.

2-2. Confirmation of Expression Levels of Present Disclosure's miRNAs Depending on Prognosis of Patients with Neurodegenerative Diseases

### \textcircled{1} The Expression Level of the Present Disclosure's

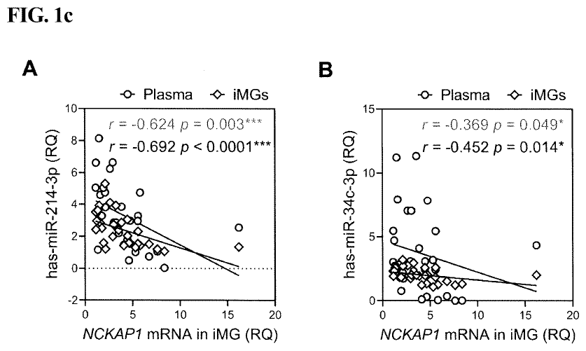

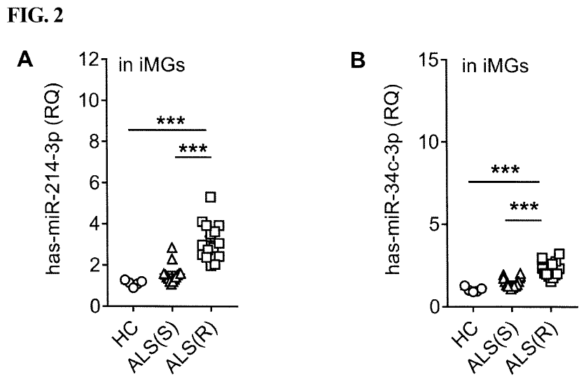

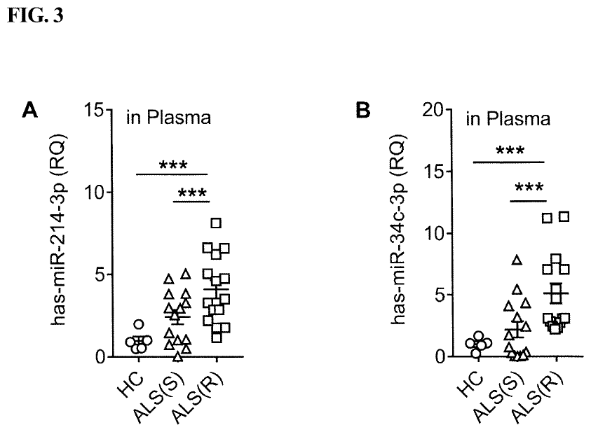

miRNAs in induced microglia-like cells in each of a normal patient group, a patient group with a slow progression rate of ASL(S), and a patient group with a rapid progression rate of ASL(R) was measured through qRT-PCR. As a result, as shown in FIG. 2, compared to the patient group showing a slow progression rate, the expression of both the miRNAs (miRNA-214-3p and miRNA-34c-3p) was observed to increase in the induced microglia-like cells in the patient group showing the fast progression rate.

\textcircled{2} The expression level of the present disclosure's miRNAs in the plasma derived from each of a normal patient group, a patient group with a slow progression of ASL(S), and a patient group with a rapid progression of ASL(R) was measured through qRT-PCR. As a result, as shown in FIG. 2, compared to the patient group showing a slow progression rate, the expression of both the miRNAs (miRNA-214-3p and miRNA-34c-3p) was observed to increase in the plasma derived from the patient group showing the fast progression rate.

Through the results, the present inventors have confirmed that the expression level of the miRNAs varies depending on the prognosis of neurodegenerative diseases, thereby the miRNAs enabling to be usefully used to predict the prognosis of neurodegenerative diseases.

2-3. Confirmation of Correlation Between Expression of Each of the Present Disclosure's miRNAs

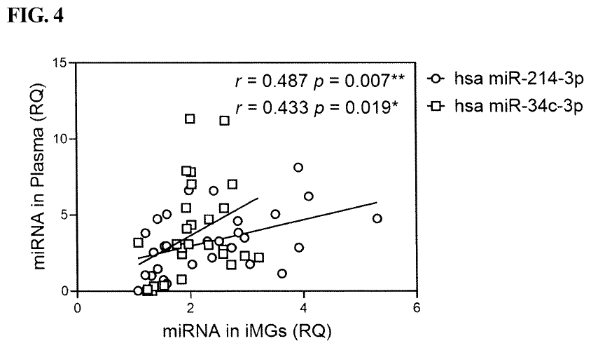

Based on the data confirmed in 2-2, the correlation between the expression of each of the miRNAs was confirmed, as measured in induced microglia-like cells (iMGs) and plasma derived from patients with ALS, respectively. As a result, as shown in FIG. 4, there was a positive correlation between the expression levels of each of the miRNAs (miRNA-214-3p and miRNA-34c-3p).

### Example 3. Verification of Prognostic Effect of Present Disclosure's miRNA on Neurodegenerative Diseases

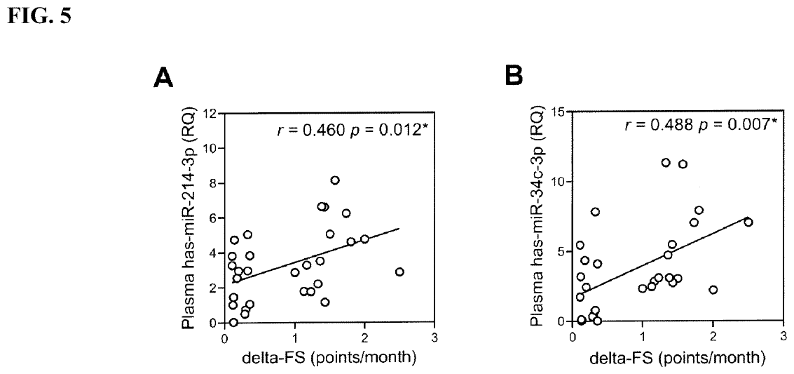

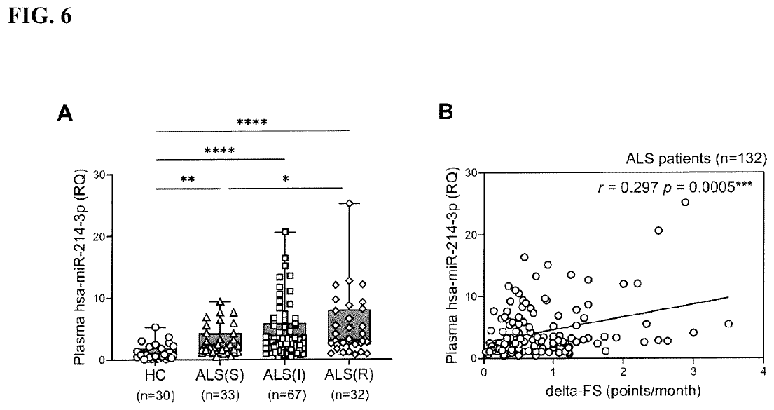

To verify the effect of the present disclosure's miRNAs in predicting the prognosis of neurodegenerative diseases, the correlation was confirmed by comparing the expression level of the present disclosure's miRNAs in plasma derived from patients with ALS and delta-FS which was an indicator of the disease progression rate. As a result, as shown in FIG. 5, the miRNAs (miRNA-214-3p and miRNA-34c-3p) showed high relative expression levels as the progression rate of the diseases increased (in other words, as the delta-FS value increased), thereby the progression rate of neurodegenerative diseases could be predicted by using the miRNAs.

### Example 4. Re-Verification of Correlation Between Expression of Present Disclosure's miRNAs and Progression Rate of Neurodegenerative Diseases

### 4-1. Recruitment of Analysis Subjects for Revalidation

A validation test was performed to confirm whether the results of Examples 2 and 3 were valid. The re-validation test was conducted on 132 patients with ALS registered on the ALS Functional Rating Scale for 6 months or more among patients who were initially registered in the ALS/MND registry database from June 2014 to May 2016 and on 30 healthy people. The patient group was divided into four groups (Q1, Q2, Q3, and Q4) based on the registered data about ALS disease progression rate, and specific data for patients are shown in Table 3 below.

TABLE 3

| | Rapid- | | | | Slow- | | | | | |
|---|---|---|---|---|---|---|---|---|---|---|
| | ALS | | Intermediate- | | ALS | | Total- | | | |
| Characteristic | (Q1) | | ALS (Q2, Q3) | | (Q4) | | ALS | | Controls | |
| Number of subjects | 32 | | 67 | | 33 | | 132 | | 30 | |
| Male:Female, n | 21:11 | | 39:28 | | 16:17 | | 76:56 | | 15:15 | |
| Onset age, yr | 60.7 | (8.9) | 57.9 | (11.0) | 56.6 | (10.9) | 58.2 | (10.5) | — | |
| Age at bio-banking, | 61.6 | (9.0) | 59.3 | (10.9) | 59.0 | (11.1) | 59.8 | (10.5) | 59.6 | (6.4) |

| yr | | | | | | | | | |
|---|---|---|---|---|---|---|---|---|---|
| ALSFRS-R at bio- | 36.3 | (5.6) | 38.7 | (5.3) | 41.2 | (4.5) | 38.8 | (5.4) | — |
| banking | | | | | | | | | |
| Duration (from | 10.3 | (8.0) | 16.4 | (14.0) | 28.4 | (33.2) | 17.9 | (20.6) | — |
| onset to bio- | | | | | | | | | |
| banking)^{a}, mo | | | | | | | | | |
| Delta-FS (from | 1.4 | (0.8) | 0.8 | (0.5) | 0.4 | (0.2) | 0.8 | (0.7) | — |
| onset to bio- | | | | | | | | | |
| banking)^{b}, point/mo | | | | | | | | | |
| Age at follow up | 62.2 | (9.0) | 60.0 | (10.8) | 59.6 | (11.2) | 60.4 | (10.5) | — |
| time (at least more | | | | | | | | | |
| than 6 months later | | | | | | | | | |
| after bio-banking), | | | | | | | | | |
| yr | | | | | | | | | |
| ALSFRS-R at follow | 19.1 | (9.4) | 31.5 | (6.3) | 39.4 | (4.5) | 30.5 | (9.9) | — |
| up time (from bio- | | | | | | | | | |
| banking to follow | | | | | | | | | |
| up) | | | | | | | | | |
| Duration (from bio- | 6.7 | (1.1) | 7.9 | (2.8) | 7.4 | (1.7) | 7.5 | (2.3) | — |
| banking to follow | | | | | | | | | |
| up)^{c}, mo | | | | | | | | | |
| Delta-FS (from bio- | 2.6 | (1.0) | 0.9 | (0.3) | 0.2 | (0.1) | 1.2 | (1.0) | — |

banking to follow
up)^{d}, point/mo

Data were expressed as mean (standard deviation) for continuous variables, and n (%) was expressed for categorical variables.

^{a} duration: The time from disease onset to registration in a biobank.

^{b}Progress rate was defined as delta FS (48-ALSFRS-R score when registered in biobanking)/(time elapsed (months) until registered in biobanking).

^{c} duration: Period from bio-banking registration to follow-up (at least 6 months have elapsed since bio-banking registration).

^{d}The progression rate level was defined as delta FS, and patients with ALS were categorized based on the following criteria: slow progression group (Q4: delta FS≤0.75), medium progression group (02, Q3: 0.46≤delta FS≤1.45), rapid progression group (Q1: delta FS>1.45).

This trial was conducted in accordance with the World Medical Association's Declaration of Helsinki and was approved by the Ethics Committee of Hanyang University Hospital (HYUH IRB 2013-06-012, 2017-01-043).

4-2. Re-Validation of Correlation Between Present Disclosure's miRNAs and Prognosis Prediction of Neurodegenerative Diseases

To re-validate Example 4-1, correlation analysis between the expression level of miRNA-214-3p in the plasma derived from a new patient group and the progression rate of neurodegenerative diseases was performed through a comparison of the expression level with the delta-FS value. As a result, as shown in FIG. 7, consistent with the existing results, the expression level of miRNA-214-3p showed a positive correlation as the delta-FS value increased.

Through the results, the present inventors reconfirmed that prognosis of neurodegenerative diseases could be predicted using miRNA-214-3p and miRNA-34c.

The description of the present disclosure stated above is for illustrative purposes, and those skilled in the art to which the present disclosure pertains can understand that the present disclosure can be easily modified into other specific forms without changing the technical idea or essential features of the present disclosure. Therefore, the examples described above should be construed in all respects as illustrative and not restrictive.

1. A marker composition for predicting prognosis of neurodegenerative diseases, the marker composition comprising at least one selected from the group consisting of microRNA-214 (miRNA-214) and microRNA-34c (miRNA-34c).

2. The marker composition of claim 1, wherein the miRNA-214 is represented by the base sequence of SEQ ID NO: 1.

3. The marker composition of claim 1, wherein the miRNA-34c is represented by the base sequence of SEQ ID NO: 2.

4. The marker composition of claim 1, wherein the neurodegenerative diseases are selected from the group consisting of Parkinson's disease, dementia, Alzheimer's disease, Frontotemporal dementia, Huntington's disease, and ALS.

5. The marker composition of claim 1, wherein the miRNA-214 and the miRNA-34c inhibit expression of Nck-associated protein 1 (NCKAP1).

6. A composition for predicting prognosis of neurodegenerative diseases, the composition comprising an agent for measuring an expression level of at least one miRNA selected from the group consisting of miRNA-214 and miRNA-34c.

7. The composition of claim 6, wherein the agent is a sense and antisense primer or a probe that binds to the miRNA complementarily.

8. The composition of claim 6, wherein the miRNA-214 is represented by the base sequence of SEQ ID NO: 1.

9. The composition of claim 6, wherein the miRNA-34c is represented by the base sequence of SEQ ID NO: 2.

10. The composition of claim 6, wherein the neurodegenerative diseases are selected from the group consisting of Parkinson's disease, dementia, Alzheimer's disease, Frontotemporal dementia, Huntington's disease, and ALS.

11. A kit for predicting prognosis of neurodegenerative diseases, the kit containing the composition of claim 6.

12. A method of providing information to predict prognosis of neurodegenerative diseases, the method comprising measuring an expression level of at least one selected from the group consisting of miRNA-214 and miRNA-34c in a biological sample derived from a subject.

13. The method of claim 12, further comprising determining that the progression of the neurodegenerative diseases is rapid when at least one selected from the group consisting of miRNA-214 and miRNA-34c exhibits a high expression level.

14. The method of claim 12, wherein the miRNA-214 is represented by the base sequence of SEQ ID NO: 1.

15. The method of claim 12, wherein the miRNA-34c is represented by the base sequence of SEQ ID NO: 2.

16. The method of claim 12, wherein the expression levels of the miRNA-214 and miRNA-34c are measured through at least one method selected from the group consisting of next generation sequencing (NGS), polymerase chain reaction (PCR), reverse transcription polymerase chain reaction (RT-PCR), and real-time. polymerase chain reaction (Real-time PCR), RNase protection assay (RPA), microarray, and northern blotting.
